SEQUENCE LISTING (17 sequences)

SEQ ID NO: 1 - DNA, 420 bp, Arabidopsis thaliana
atggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttcttgattcagaagaaaggaagagatag

SEQ ID NO: 2 - protein, 139 aa, Arabidopsis thaliana
MESSNRSSNNQSQDDKQARFRGVRRRPWGKFAAEIRDPSRNGARLWLGTFETAEEAARAYDRAAFNLRGHLAILNFPNEYYPRMDDYSLRPPYASSSSSSSSGSTSTNVSRQNQREVFEFEYLDDKVLEELLDSEERKR

SEQ ID NO: 3 - DNA, 687 bp, Glycine max
atggggagaggtaagatcgtgataaggaggatcgacaattccacgagcaggcaagtgacgttctcgaagcgaaggaacggtttgctgaagaaggcgaaggagcttgcgatcttgtgcgatgctgaagtcggagttatgatcttctccagcaccggaaaactctacgatttcgccagctccagcatgaaatcagtaatggaccgatacagcaaatcaaaagaagaaccttgtcaacttgggagttcagcctctgaaattaagttttggcaaagggaggcagcaatgttaaggcaacaattacacaatttgcaagaaagtcaccgcaggaaaatgatgggggaagaactgtcaggcttgacagtcaaagaattacaaaatttggagaaccaattagaaattagccttcatggtgtccgaatgaaaaaggatcaacttttaatgggtgaaatacaagagctaaatcgaaagggaaacctcatacaccaagaaaatgtggaactgtataagaaggtctatggaacacaagatgataacgaaacaaacagagattctgttctgacaaatggtctaggcataggagaggatttgcaagtgcctgtgaatctccagctaagccagccacagcaacagcaacaacactacaaggcatcttcaggaactacaaaattgggattgcaattgcattga

SEQ ID NO: 4 - protein, 228 aa, Glycine max
MGRGKIVIRRIDNSTSRQVTFSKRRNGLLKKAKELAILCDAEVGVMIFSSTGKLYDFASSSMKSVMDRYSKSKEEPCQLGSSASEIKFWQREAAMLRQQLHNLQESHRRKMMGEELSGLTVKELQNLENQLEISLHGVRMKKDQLLMGEIQELNRKGNLIHQENVELYKKVYGTQDDNETNRDSVLTNGLGIGEDLQVPVNLQLSQPQQQQQHYKASSGTTKLGLQLH

SEQ ID NO: 5 - DNA, 564 bp, Glycine max
atggccaaagaaggtcttggacttgaaatcaccgagctaaggttgggtctacccgatgcggagcatgtaactgtggtcaataagaatgagaagaagagggccttctcgcaaattgatgatgaaaatagctcctccggcggcgaccggaaaatcaagaccaataagagtcaagtggtggggtggcctccggtgtgctcttaccggaagaagaacagcatgaacgaaggttcaaagatgtacgtgaaggttagcatggacggtgctcctttcttgcgcaaaattgatttgggtctccataaggggtactcagatttagccttggctttggacaagctctttggttcctatggaatggtggaggccttgaagaatgcggacaattctgaacacgttcccatttatgaggacaaagatggtgactggatgcttgttggagatgtcccttgggaaatgtttatggagtcatgcaagaggctgaggattatgaagaggtcagatgccaagggcttcggtttgcaaccaaaaggatctctgaagggattcatagaaagcgcggcaaagtag

SEQ ID NO: 6 - protein, 187 aa, Glycine max
MAKEGLGLEITELRLGLPDAEHVTVVNKNEKKRAFSQIDDENSSSGGDRKIKTNKSQVVGWPPVCSYRKKNSMNEGSKMYVKVSMDGAPFLRKIDLGLHKGYSDLALALDKLFGSYGMVEALKNADNSEHVPIYEDKDGDWMLVGDVPWEMFMESCKRLRIMKRSDAKGFGLQPKGSLKGFIESAAK

SEQ ID NO: 7 - DNA, 801 bp, Arabidopsis thaliana
atggggagacagccatgctgtgacaagctaggggtgaagaaagggccgtggacggtggaggaagataagaagcttataaacttcatactaaccaatggccattgttgctggcgtgctttgccgaagctggccggtctccgtcgctgtggaaagagctgccgcctccggtggactaactatctccggcctgacttaaaacgaggccttctctcgcatgatgaagaacaacttgtcatagatcttcatgctaatctcggcaataagtggtctaagatagcttcaagattacctggaagaacagataacgaaataaaaaaccattggaatactcatatcaagaagaaacttcttaagatgggaatcgatcctatgacccatcaacccctaaatcaagaaccttctaatatcgataattccaaaaccattccgtccaatccagacgatgtctcagtggaaccaaagacaactaacacgaaatacgtggagataagtgtcacgacaacagaagaagaaagtagtagcacggttactgatcaaaacagttcgatggataatgaaaatcatctaattgacaacatttatgatgatgatgaattgtttagttacttatggtccgacgaaactactaaagatgaggcctcttggagtgatagtaactttggtgttggtggaacattatatgaccacaatatctccggcgccgatgcagattttccgatatggtcaccggaaagaatcaatgacgagaagatgtttttggattattgtcaagactttggtgttcatgattttgggttttag

SEQ ID NO: 8 - protein, 266 aa, Arabidopsis thaliana
MGRQPCCDKLGVKKGPWTVEEDKKLINFILTNGHCCWRALPKLAGLRRCGKSCRLRWTNYLRPDLKRGLLSHDEEQLVIDLHANLGNKWSKIASRLPGRTDNEIKNHWNTHIKKKLLKMGIDPMTHQPLNQEPSNIDNSKTIPSNPDDVSVEPKTTNTKYVEISVTTTEEESSSTVTDQNSSMDNENHLIDNIYDDDELFSYLWSDETTKDEASWSDSNFGVGGTLYDHNISGADADFPIWSPERINDEKMFLDYCQDFGVHDFGF

SEQ ID NO: 9 - DNA, 1446 bp, Zea mays
atggacttctcagctggttcctacttcacatcatggcctgtcaattctgcttctgagagctacagtttggctgatggttcagttgaatcctatggaggagaaggaatcatgccaccttcaagctacttcatgacagccagatcagatcacaatttaaaattcagtgtgcatgaacaggattccactatgcttccaaacgaccaattgacctatgctggcgctaggcagactgatctattacctggcgagactccatctagggataagctttgtgagaatcttctggagcttcaacgactgcagaataacagcagcctaccgagtaatttagtgcccccaggggtacttcagcacaattcaacacctggggcctttcatccgcagttgaatactcctggactttcagaactgcctcatgccttatctagttcaattgatagtaatggaagtgaagtttctgcttttctcgctgatctaaatgctgtttcttcagcctcagctttgtgttccacattccaaaatgcttcttcattcatggaaccagtaaatctagaagctttcagttttcaaggggcacaaagtgattctgttttgaacaaaacaacacatccaaatgggaatatctcagtatttgacagtgctgccttggcatcactacatgatagcaaagagtttatcagtggtaggctcccctcatttgctagtgtccaggaaacaaacgtagctgctagtggtttcaagactcagaagcaggagcaaaatgcagtgtgcaacgttcctatccctacgttcactgcacgtaatcaaattgcagttgctgcaatgcccggatcactgatccctcaaaagattccttcatggatcaatgaaaacaaaagtgagggtcctgttagccatccttctgatgtacaaatccaaccaaattctgttggaaatggtgttggtgtgaagccacgagtaagggctcgtcgtggacaggcaactgatcctcatagcattgctgaacgacttcgcagagaaaaaatttcagataggatgaaaaatctacaagatcttgtcccgaattctaataaggcagacaaggcatccatgctggatgaaatcattgatcatgtgaaatttcttcagcttcaggtcaaggtgttaagcatgagtaggctaggagctccaggagcagttcttccccttctcgcagaatctcagactgagggttaccgcggtcaacttctgtcagctccaaccaacgcacaaggattactggacacggaagaatcggaagacaccttcgccttcgaggaagaggttgtgaagctaatggaaaccagcatcacaagcgcaatgcagtaccttcagaacaagggactctgcctgatgccagttgcccttgcttccgccatatccacccagaagggcgtctccgccgcgtcaatccctcccgaacagtag

SEQ ID NO: 10 - protein, 481 aa, Zea mays
MDFSAGSYFTSWPVNSASESYSLADGSVESYGGEGIMPPSSYFMTARSDHNLKFSVHEQDSTMLPNDQLTYAGARQTDLLPGETPSRDKLCENLLELQRLQNNSSLPSNLVPPGVLQHNSTPGAFHPQLNTPGLSELPHALSSSIDSNGSEVSAFLADLNAVSSASALCSTFQNASSFMEPVNLEAFSFQGAQSDSVLNKTTHPNGNISVFDSAALASLHDSKEFISGRLPSFASVQETNVAASGFKTQKQEQNAVCNVPIPTFTARNQIAVAAMPGSLIPQKIPSWINENKSEGPVSHPSDVQIQPNSVGNGVGVKPRVRARRGQATDPHSIAERLRREKISDRMKNLQDLVPNSNKADKASMLDEIIDHVKFLQLQVKVLSMSRLGAPGAVLPLLAESQTEGYRGQLLSAPTNAQGLLDTEESEDTFAFEEEVVKLMETSITSAMQYLQNKGLCLMPVALASAISTQKGVSAASIPPEQ

SEQ ID NO: 11 - DNA, 2502 bp, Arabidopsis thaliana
atgggaggaggaagcaataatagtcacaatatggacaacgggaagtacgtgaggtacactcctgaacaagtggaagctctggagagactctacaatgactgtcctaaaccgagctctatgcgccgccaacagctaatccgcgaatgtcctatcctctccaacatcgagcctaaacagataaaagtctggttccagaaccgcaggtgtagagaaaaacagcgaaaagaggcgtcacgacttcaagctgtgaataggaagctaacggcaatgaacaagcttttgatggaagagaatgaccgcttgcaaaagcaagtgtctcacttggtttatgagaacagctattttcgccaacatcctcaaaaccaggggaatttggctaccacagatactagctgtgagtcagtggtgacgagtggtcaacaccacttgacccctcaacatcagcctcgtgatgctagtcctgctggattattgtccattgcggatgaaactttaacagagttcatttccaaggcgactggaaccgctgtcgagtgggtccaaatgcctgggatgaagcctggtccggattccataggaatcgttgctatttctcatggatgcacgggaatcgctgctcgtgcttgcggccttgtgggtcttgatcccacaagggtcgcggagatcctaaaggataagccttgttggttgcgtgattgcagatctctggatatagttaacgtactatccactgcaaatggtggaacccttgaactaatctacatgcagctttatgcgccgacaacactggcaccagctcgtgacttctggatgctacgttacacatctgtaatggaagacgggagccttgtgatatgcgaacgatcactgaacaatacacaaaacgggccaagtatgcctccgtctcctcatttcgttagggcagagattttaccaagtggttacctcattagaccttgcgaaggaggtggatccattcttcacattgtcgatcatttcgatcttgagccatggagtgtgccagaagttcttcgttctctctatgagtcctccactttactcgcccaaagaactacaatggccgctctgcgctatttgaggcaaatatctcaagagatttcacaacctaacgtaacaggttggggaagaagaccagcggctcttagagcacttagccaaaggcttagcaaaggattcaacgaagcagtgaatgggttcagcgatgaaggatggtccatcctagagagtgatggtatcgatgatgtcactcttcttgtgaactcctctcccacaaagatgatgatgacttcaagtctcccatttgccaatggctacacttctatgcctagcgcggtcttatgtgccaaagcttccatgttattacaaaatgttccaccttcgattctgctgcggttcttgagggaacatagacaagaatgggcagacaatagcatcgatgcatattcggccgcagccatcaaagcaggcccttgtagcttaccaatccctcgcccagggagctttggtggtcaagtcattcttcctctagctcacactatagagaatgaagagtttatggaagtcattaagcttgagagcttggggcactaccaagaagacatgatgatgcctgctgatatcttccttctgcaaatgtgcagtggggtggatgagaacgcagttgaatcatgcgcagagcttatatttgcaccaatcgacgcatctttctctgatgatgcaccaatcattccttccggtttccgcatcattcctctagattccaaatcagaggggttgagtcctaaccgaacgctagacctagcgtcggctctagacgtagggagcagaacagccggagattcatgtggaagcagaggaaactcaaagtccgttatgactatagcgtttcagctagcttttgagatgcatatgcaagagaatgtagcctcaatggctagacagtatgtaagaagtgtgatcgcgtcggttcaacgggtcgcacttgctctctctccttcttctcatcagctaagtggcttgcgtcctccacccgcgtcacccgaagctcacactctcgctcgctggatctctcactcttatagatgttaccttggcgttgatcttctcaaacctcatggaactgatcttctcaagtctctttggcaccatcctgacgctgtcatgtgttgctcactcaaggccttatctccggtattcacatttgcgaaccaagctggtttagacatgctggagacgacgttggtggcacttcaagatatcactctcgacaagatcttcgacaacaacaacgggaagaagactttatcctccgaattccctcagatcatgcaacaggggtttatgtgtatggatggaggaatatgcatgtcgagcatgggaagagcagtaacgtacgagaaggctgttgggtggaaagtgttgaacgacgacgaagatcctcattgtatctgcttcatgttcctcaactggtcttttatatag

SEQ ID NO: 12 - protein, 833 aa, Arabidopsis thaliana
MGGGSNNSHNMDNGKYVRYTPEQVEALERLYNDCPKPSSMRRQQLIRECPILSNIEPKQIKVWFQNRRCREKQRKEASRLQAVNRKLTAMNKLLMEENDRLQKQVSHLVYENSYFRQHPQNQGNLATTDTSCESVVTSGQHHLTPQHQPRDASPAGLLSIADETLTEFISKATGTAVEWVQMPGMKPGPDSIGIVAISHGCTGIAARACGLVGLDPTRVAEILKDKPCWLRDCRSLDIVNVLSTANGGTLELIYMQLYAPTTLAPARDFWMLRYTSVMEDGSLVICERSLNNTQNGPSMPPSPHFVRAEILPSGYLIRPCEGGGSILHIVDHFDLEPWSVPEVLRSLYESSTLLAQRTTMAALRYLRQISQEISQPNVTGWGRRPAALRALSQRLSKGFNEAVNGFSDEGWSILESDGIDDVTLLVNSSPTKMMMTSSLPFANGYTSMPSAVLCAKASMLLQNVPPSILLRFLREHRQEWADNSIDAYSAAAIKAGPCSLPIPRPGSFGGQVILPLAHTIENEEFMEVIKLESLGHYQEDMMMPADIFLLQMCSGVDENAVESCAELIFAPIDASFSDDAPIIPSGFRIIPLDSKSEGLSPNRTLDLASALDVGSRTAGDSCGSRGNSKSVMTIAFQLAFEMHMQENVASMARQYVRSVIASVQRVALALSPSSHQLSGLRPPPASPEAHTLARWISHSYRCYLGVDLLKPHGTDLLKSLWHHPDAVMCCSLKALSPVFTFANQAGLDMLETTLVALQDITLDKIFDNNNGKKTLSSEFPQIMQQGFMCMDGGICMSSMGRAVTYEKAVGWKVLNDDEDPHCICFMFLNWSFI

SEQ ID NO: 13 - protein, 140 aa, Arabidopsis lyrata
MESSNRRSNNQSQDDKQARFRGVRRRPWGKFAAEIRDPSRNGARLWLGTFETAEEAARAYDRAAYNLRGHLAILNFPNEYYSRMDDYSLRPPYASSSSSSSSSGSTSTNASRQNQREVFEFEYLDDRVLEELLDSEERKR

SEQ ID NO: 14 - protein, 209 aa, unknown
  ortholog to Glycine max sequence
MAHHHHHHSAAEQKLISEEDLVAAKEGLGLEITELRLGLPDAEHVTVVNKNEKKRAFSQIDDENSSSGGDRKIKTNKSQVVGWPPVCSYRKKNSMNEGSKMYVKVSMDGAPFLRKIDLGLHKGYSDLALALDKLFGSYGMVEALKNADNSEHVPIYEDKDGDWMLVGDVPWEMFMESCKRLRIMKRSDAKGFGLQPKGSLKGFIESAAK

SEQ ID NO: 15 - protein, 481 aa, Zea mays
MDFSAGSYFSSWPVNSASESYSLADGSVESYGGEGIMPPSSYFMTARSDHNLKFSVHEQDSTMLPNDQLTYAGARQTDLLPGETPSRDKLCENLLELQRLQNNSNLPSNLVPPGVLQHNSTPGAFHPQLNTPGLSELPHALSSSIDSNGSEVSAFLADLNAVSSASALCSTFQNASSFMEPVNLEAFSFQGAQSDSVLNKTTHPDGNISVFDSAALASLHDSKEFISGRLPSFASVQETNVAASGFKTQKQEQNAVCNVPIPTFTARNQIAVAAMPGSLIPQKIPSWINENKSEGPVSHPSDVQIQPNSVGNGVGVKPRVRARRGQATDPHSIAERLRREKISDRMKNLQDLVPNSNKADKASMLDEIIDYVKFLQLQVKVLSMSRLGAPGAVLPLLAESQTEGYRGQLLSAPTNAQGLLDTEESEDTFAFEEEVVKLMETSITSAMQYLQNKGLCLMPVALASAISTQKGVSAASIPPEQ

SEQ ID NO: 16 - protein, 481 aa, Zea mays; identical to SEQ ID NO: 10

SEQ ID NO: 17 - protein, 481 aa, Zea mays
MDFSAGSYFSSWPVNSASESYSLADGSVESYGGEGIMPPSSYFMAARSDHNLKFSVHEQDSTMLPNDQLTYAGARQTDLLPGETPSRDKLCENLLELQRLQNNSNLPSNLVPPGVLQHNSTPGAFHPQLNTPGLSELPHALSSSIDSNGSEVSAFLADLNAVSSASALCSTFQNASSFMEPVNLEAFSFQGAQSDSVLNKTTHPDGNISVFDSAALASLHDSKEFISGRLPSFASVQETNVAASGFKTQKREQNAVCNVPIPTFTARNQIAVAAMPGSLIPQKIPSWINENKSEGPVSHPSDVQIQPNSVGNGVGVKPRVRARRGQATDPHSIAERLRREKISDRMKNLQDLVPNSNKADKASMLDEIIDYVKFLQLQVKVLSMSRLGAPGAVLPLLAESQTEGYRGQLLSAPTNAQGLLDTEESEDTFAFEEEVVKLMETSITSAMQYLQNKGLCLMPVALASAISTQKGVSAASIPPEQ

(19) United States
(12) Patent Application Publication
(10) Pub. No.: US 2015/0344899 A1
(43) Pub. Date: Dec. 3, 2015

(54) Transgenic Plants With Enhanced Traits

(71) Applicant: MONSANTO TECHNOLOGY LLC, St. Louis, MO (US)

(72) Inventors: Mark S. Abad, Webster Groves, MO (US); Paul S. Chomet, St. Louis, MO (US); Dhanalakshmi Ramachandra, Bangalore (IN); Tyamagondlu V. Venkatesh, St. Louis, MO (US); Xiaoyun Wu, Chesterfield, MO (US)

(21) Appl. No.: 14/722,303

(22) PCT Filed: Mar. 6, 2013
(86) PCT No.: PCT/US13/29243

§ 371 Date: May 27, 2015

Related U.S. Application Data
(60) Provisional application No. 61/730,731, filed on Nov. 28, 2012.

(51) Int. Cl. C12N 15/82

(52) U.S. Cl. CPC C12N 15/8261

(57) ABSTRACT

This disclosure provides plants having enhanced traits such as increased yield, increased nitrogen use efficiency and increased water use efficiency; propagules, progeny and field crops of such transgenic plants; and methods of making and using such transgenic plants. This disclosure also provides methods of producing hybrid seed from such transgenic plants, growing such seed and selecting progeny plants with the composition or with enhanced traits.

### CROSS REFERENCE TO RELATED APPLICATIONS

This application claims the benefit under 35USC §119(e) of U.S. provisional application Ser. No. 61/730,731, filed on Nov. 28, 2012, and is herein incorporated by reference in its entirety.

### INCORPORATION OF SEQUENCE LISTING

The sequence listing file named “38-21_{—}59495_B.txt”, which is 43,044 bytes (measured in MS-WINDOWS) and was created on Mar. 5, 2013, is filed herewith and incorporated herein by reference in its entirety.

### FIELD OF THE INVENTION

Disclosed herein are plants having enhanced traits such as increased yield, increased nitrogen use efficiency and increased water use efficiency; propagules, progenies and field crops of such plants; and methods of making and using such plants. Also disclosed are methods of producing seed from such plants, growing such seed and/or selecting progeny plants with enhanced traits.

### SUMMARY OF THE INVENTION

An aspect of this disclosure provides a plant comprising a recombinant DNA molecule comprising a polynucleotide encoding a polypeptide, wherein the nucleotide sequence of the polynucleotide is selected from the group consisting of: a) a nucleotide sequence encoding a protein having the amino acid sequence of SEQ ID NO: 2, 4, 6, 8, 10, 12, 13, 14, 15, 16, or 17; and b) a nucleotide sequence encoding a protein with at least 90%, at least 91%, at least 92%, at least 93%, at least 94%, at least 95%, at least 96%, at least 97%, at least 98%, at least 99% identity to SEQ ID NO: 2, 4, 6, 8, 10, 12, 13, 14, 15, 16, or 17; and wherein the plant has at least one enhanced trait as compared to a control plant.

Another aspect of this disclosure also provides a plant, wherein the plant has at least one enhanced trait as compared to a control plant, and wherein the enhanced trait is selected from the group consisting of increased yield, increased nitrogen use efficiency and increased water use efficiency.

Another aspect of this disclosure provides a plant comprising a recombinant DNA molecule of the disclosure, wherein said plant is a monocot plant or is a member of the family Poaceae, wheat plant, maize plant, sweet corn plant, rice plant, wild rice plant, barley plant, rye, millet plant, sorghum plant, sugar cane plant, turfgrass plant, bamboo plant, oat plant, brome-grass plant, Miscanthus plant, pampas grass plant, switchgrass (Panicum) plant, and/or teosinte plant, or is a member of the family Alliaceae, onion plant, leek plant, garlic plant; or wherein the plant is a dicot plant or is a member of the family Musaceae, banana plant, or is a member of the family Amaranthaceae, spinach plant, quinoa plant, a member of the family Anacardiaceae, mango plant, a member of the family Asteraceae, sunflower plant, endive plant, lettuce plant, artichoke plant, a member of the family Brassicaceae, Arabidopsis thaliana plant, rape plant, oilseed rape plant, broccoli plant, Brussels sprouts plant, cabbage plant, canola plant, cauliflower plant, kohlrabi plant, turnip plant, radish plant, a member of the family Bromeliaceae, pineapple plant, a member of the family Caricaceae, papaya plant, a member of the family Chenopodiaceae, beet plant, a member of the family Curcurbitaceae, melon plant, cantaloupe plant, squash plant, watermelon plant, honeydew plant, cucumber plant, pumpkin plant, a member of the family Dioscoreaceae, yam plant, a member of the family Ericaceae, blueberry plant, a member of the family Euphorbiaceae, cassava plant, a member of the family Fabaceae, alfalfa plant, clover plant, peanut plant, a member of the family Grossulariaceae, currant plant, a member of the family Juglandaceae, walnut plant, a member of the family Lamiaceae, mint plant, a member of the family Lauraceae, avocado plant, a member of the family Leguminosae, soybean plant, bean plant, pea plant, a member of the family Malvaceae, cotton plant, a member of the family Marantaceae, arrowroot plant, a member of the family Myrtaceae, guava plant, eucalyptus plant, a member of the family Rosaceae, peach plant, apple plant, cherry plant, plum plant, pear plant, prune plant, blackberry plant, raspberry plant, strawberry plant, a member of the family Rubiaceae, coffee plant, a member of the family Rutaceae, citrus plant, orange plant, lemon plant, grapefruit plant, tangerine plant, a member of the family Salicaceae, poplar plant, willow plant, a member of the family Solanaceae, potato plant, sweet potato plant, tomato plant, Capsicum plant, tobacco plant, tomatillo plant, eggplant plant, Atropa belladona plant, Datura stramonium plant, a member of the family Vitaceae, grape plant, a member of the family Umbelliferae, carrot plant, or a member of the family Musaceae; or wherein the plant is a member of the family Pinaceae, cedar plant, fir plant, hemlock plant, larch plant, pine plant, or spruce plant.

Another aspect of this disclosure provides a plant comprising a recombinant DNA molecule of the disclosure, wherein the recombinant DNA molecule further comprises a promoter that is operably linked to the polynucleotide encoding a polypeptide, wherein said promoter is selected from the group consisting of a constitutive, inducible, tissue specific, diurnally regulated, tissue enhanced, and cell specific promoter.

Another aspect of this disclosure provides a plant comprising a recombinant DNA molecule of the disclosure, wherein said plant is a progeny, propagule, or field crop. Such field crop is selected from the group consisting of corn, soybean, cotton, canola, rice, barley, oat, wheat, turf grass, alfalfa, sugar beet, sunflower, quinoa and sugar cane.

Another aspect of this disclosure provides a plant comprising a recombinant DNA molecule of the disclosure, wherein said plant is a progeny, propagule, or field crop. Such propagule is selected from the group consisting of a cell, pollen, ovule, flower, embryo, leaf, root, stem, shoot, meristem, grain and seed.

Another aspect of this disclosure provides a method for producing a plant comprising: introducing into a plant cell a recombinant DNA comprising a polynucleotide encoding a polypeptide, wherein the nucleotide sequence of the polynucleotide is selected from the group consisting of: a) a nucleotide sequence encoding a protein having the amino acid sequence of SEQ ID NO: 2, 4, 6, 8, 10, 12, 13, 14, 15, 16 or 17; and b) a nucleotide sequence encoding a protein with at least 90%, at least 91%, at least 92%, at least 93%, at least 94%, at least 95%, at least 96%, at least 97%, at least 98%, at least 99% identity to SEQ ID NO: 2, 4, 6, 8, 10, 12, 13, 14, 15, 16 or 17; and growing a plant from the plant cell.

Another aspect of this disclosure provides a method further comprising selecting a plant comprising the recombinant DNA molecule with at least one enhanced trait selected from a group consisting of increased yield, increased nitrogen use efficiency and increased water use efficiency as compared to a control plant.

Another aspect of this disclosure provides a method of increasing yield, increasing nitrogen use efficiency, or increasing water use efficiency in a plant comprising: a) crossing the plant with itself, a second plant from the same plant line, a wild type plant, or a second plant from a different line of plants to produce a seed; b) growing the seed to produce a plurality of progeny plants, and c) selecting a progeny plant with increased yield, increased nitrogen use efficiency, or increased water use efficiency compared to a plant not having the recombinant DNA molecule.

### DETAILED DESCRIPTION OF THE INVENTION

In the attached sequence listing:

SEQ ID NOs: 1, 3, 5, 7, 9, and 11 are nucleotide sequences of the coding strand of the DNA molecules used in the recombinant DNA imparting at least one enhanced trait in plants, each represent a coding sequence for a protein.

SEQ ID NOs: 2, 4, 6, 8, 10, and 12 are amino acid sequences of the cognate proteins of the DNA molecules with nucleotide sequences 1, 3, 5, 7, 9, and 11.

SEQ ID NO: 13, 14, 15, 16, and 17 are amino acid sequences of orthologous proteins.

As used herein, the term “expression” refers to the activity level of a gene in a plant, plant cell or plant tissue in producing a protein. Expression is the process by which information from a gene is used in the synthesis of a functional gene product. Gene expression can give rise to the phenotype. Such phenotypes are often expressed by the synthesis of proteins that control the organism's shape, or that acts as enzymes catalyzing specific metabolic pathways. “Expression or altered expression” in reference to a polynucleotide indicates that the pattern of expression in, for example, a transgenic plant or plant tissue, is different from the expression pattern in a wild-type plant or a non-transgenic plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” can relate to altered activity levels resulting from the interactions of the polypeptides with exogenous or endogenous modulators or from interactions with factors or as a result of the chemical modification of the polypeptides. Variation in expression can occur when, for example, the genes encoding one or more polypeptides are under the control of a constitutive promoter (for example, the cauliflower mosaic virus 35S transcription initiation region). Expression can also be altered by having the gene under the control of an endogenous or a heterologous promoter, or an inducible or tissue specific promoter. Expression can occur throughout a plant, in specific tissues of the plant, or in the presence or absence of particular environmental signals, depending on the promoter used. Expression can also occur in plant cells where endogenous expression of the present polypeptides or functionally equivalent molecules normally occurs, but such normal expression is at a lower level.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more polypeptides are under the control of a promoter (for example, the cauliflower mosaic virus 35S transcription initiation region). Overexpression can also be under the control of a heterologous promoter, or an inducible or tissue specific promoter. Thus, overexpression can occur throughout a plant, in specific tissues of the plant, or in the presence or absence of particular environmental signals, depending on the promoter used. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

The term “suppression” as used herein refers to a lower expression level of a gene in a plant, plant cell or plant tissue, compared to the expression in a wild-type or control plant, cell or tissue, at any developmental or temporal stage for the gene. Suppression can be applied using numerous approaches. Non limiting examples include: to suppress an endogenous gene(s) or a subset of genes in a pathway, to suppress a mutation that has resulted in decreased activity of a protein, to suppress the production of an inhibitory agent, to elevate, reduce or eliminate the level of substrate that an enzyme requires for activity, to produce a new protein, to activate a normally silent gene; or to accumulate a product that does not normally increase under natural conditions.

As used herein a “plant” includes whole plant, transgenic plant, meristem, shoot organ/structure (for example, leaf, stem and tuber), root, flower and floral organ/structure (for example, bract, sepal, petal, stamen, carpel, anther and ovule), seed (including embryo, endosperm, and seed coat) and fruit (the mature ovary), plant tissue (for example, vascular tissue, ground tissue, and the like) and cell (for example, guard cell, egg cell, pollen cell, mesophyll cell, and the like), and progeny of same. The classes of plants that can be used in the disclosed methods are generally as broad as the classes of higher and lower plants amenable to transformation and breeding techniques, including angiosperms (monocotyledonous and dicotyledonous plants), gymnosperms, ferns, horsetails, psilophytes, lycophytes, bryophytes, and algae.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transgenic plant.

As used herein a “control plant” means a plant that does not contain the recombinant DNA that imparts an enhanced trait. A control plant is used to identify and select a transgenic plant that has an enhanced trait. A suitable control plant can be a non-transgenic plant of the parental line used to generate a transgenic plant, for example, a wild type plant devoid of a recombinant DNA. A suitable control plant can also be a transgenic plant that contains the recombinant DNA that imparts other traits, for example, a transgenic plant having enhanced herbicide tolerance. A suitable control plant can in some cases be a progeny of a hemizygous transgenic plant line that does not contain the recombinant DNA, known as a negative segregant, or a negative isoline.

As used herein a “transgenic plant cell” means a plant cell that is transformed with stably-integrated, recombinant DNA, for example, by Agrobacterium-mediated transformation or by bombardment using microparticles coated with recombinant DNA or by other means. A plant cell of this disclosure can be an originally-transformed plant cell that exists as a microorganism or as a progeny plant cell that is regenerated into differentiated tissue, for example, into a transgenic plant with stably-integrated, recombinant DNA, or seed or pollen derived from a progeny transgenic plant.

As used herein a “propagule” includes all products of meiosis and mitosis, including but not limited to, plant, seed and part of a plant able to propagate a new plant. Propagules include whole plants, cells, pollen, ovules, flowers, embryos, leaves, roots, stems, shoots, meristems, grains or seeds, or any plant part that is capable of growing into an entire plant. Propagule also includes graft where one portion of a plant is grafted to another portion of a different plant (even one of a different species) to create a living organism. Propagule also includes all plants and seeds produced by cloning or by bringing together meiotic products, or allowing meiotic products to come together to form an embryo or a fertilized egg (naturally or with human intervention).

As used herein a “progeny” includes any plant, seed, plant cell, and/or regenerable plant part comprising a recombinant DNA of the present disclosure derived from an ancestor plant. A progeny can be homozygous or heterozygous for the transgene. Progeny can be grown from seeds produced by a transgenic plant comprising a recombinant DNA of the present disclosure, and/or from seeds produced by a plant fertilized with pollen or ovule from a transgenic plant comprising a recombinant DNA of the present disclosure.

As used herein a “trait” is a physiological, morphological, biochemical, or physical characteristic of a plant or particular plant material or cell. In some instances, this characteristic is visible to the human eye, such as seed or plant size, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants.

As used herein an “enhanced trait” means a characteristic of a transgenic plant as a result of stable integration and expression of a recombinant DNA in the transgenic plant. Such traits include, but are not limited to, an enhanced agronomic trait characterized by enhanced plant morphology, physiology, growth and development, yield, nutritional enhancement, disease or pest resistance, or environmental, or chemical tolerance. In some specific aspects of this disclosure an enhanced trait is selected from the group consisting of increased yield, increased nitrogen use efficiency, and increased water use efficiency, or drought tolerance as shown in Tables 2-7. In another aspect of the disclosure the trait is increased yield under non-stress conditions or increased yield under environmental stress conditions. Stress conditions can include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, plant biomass, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), car biomass, ear biomass per plot, ear number, seed number per car, seed size, composition of seed (starch, oil, protein) and characteristics of seed fill.

Also used herein, the term “trait modification” encompasses altering the naturally occurring trait by producing a detectable difference in a characteristic in a plant comprising a recombinant DNA encoding a polypeptide of the present disclosure relative to a plant not comprising the recombinant DNA, such as a wild-type plant, or a negative segregant. In some cases, the trait modification can be evaluated quantitatively. For example, the trait modification can entail an increase or decrease, in an observed trait as compared to a control plant. It is known that there can be natural variations in the modified trait. Therefore, the trait modification observed entails a change of the normal distribution and magnitude of the trait in the plants as compared to a control plant.

Increased yield of a plant of the present disclosure can be measured in a number of ways, including test weight, seed number per plant, seed weight, seed number per unit area (for example, seeds, or weight of seeds, per acre), bushels per acre, tons per acre, or kilo per hectare. For example, corn yield can be measured as production of shelled corn kernels per unit of production area, for example in bushels per acre or metric tons per hectare. Increased yield can result from improved utilization of key biochemical compounds, such as nitrogen, phosphorous and carbohydrate, or from improved responses to environmental stresses, such as cold, heat, drought, salt, shade, high plant density, and attack by pests or pathogens. This disclosure can also be used to provide plants with improved growth and development, and ultimately increased yield, as the result of modified expression of plant growth regulators or modification of cell cycle or photosynthesis pathways. Also of interest is the generation of plants that demonstrate increased yield with respect to a seed component that may or may not correspond to an increase in overall plant yield.

The present disclosure relates to a plant with improved economically important characteristics, more specifically increased yield. More specifically the present disclosure relates to a plant comprising a polynucleotide of this disclosure that encodes a polypeptide, wherein the plant has increased yield as compared to a control plant. Many plants of this disclosure exhibited increased yield as compared to a control plant. In an embodiment, a plant of the present disclosure exhibited an improved trait that is a component of yield.

Yield can be defined as the measurable produce of economic value from a crop. Yield can be defined in the scope of quantity and/or quality. Yield can be directly dependent on several factors, for example, the number and size of organs, plant architecture (such as the number of branches, plant biomass, etc.), seed production and more. Root development, photosynthetic efficiency, nutrient uptake, stress tolerance, early vigor, delayed senescence and functional stay green phenotypes can be important factors in determining yield. Optimizing the above mentioned factors can therefore contribute to increasing crop yield.

Reference herein to an increase in yield-related traits can also be taken to mean an increase in biomass (weight) of one or more parts of a plant, which can include above ground and/or below ground (harvestable) plant parts. In particular, such harvestable parts are seeds, and performance of the methods of the disclosure results in plants with increased yield and in particular increased seed yield relative to the seed yield of suitable control plants. The term “yield” of a plant can relate to vegetative biomass (root and/or shoot biomass), to reproductive biomass (ear biomass or ear biomass per plot), and/or to propagules (such as seeds) of that plant.

In an embodiment, “alfalfa yield” can also be measured in forage yield, the amount of above ground biomass at harvest. Factors contributing to increased biomass include increased vegetative growth, branches, nodes and internodes, leaf area, and leaf area index.

In another embodiment, “canola yield” can also be measured in silique number, number of siliques per plant, number of siliques per node, number of internodes, incidence of silique shatter, seeds per silique, seed weight per silique, improved seed, oil, or protein composition.

Additionally, “corn or maize yield” can also be measured as the production of shelled corn kernels per unit of production area, ears per acre, number of kernel rows per ear, kernels per row, weight per kernel, ear number, ear biomass and ear biomass per plot.

In yet another embodiment, “cotton yield” can be measured as bolls per plant, size of bolls, fiber quality, seed cotton yield in grams per plant, seed cotton yield in pounds per acre, lint yield in pounds per acre, and number of bales.

Specific embodiment for “rice yield” can also include panicles per hill, grain per hill, and filled grains per panicle.

Still further embodiment for “soybean yield” can also include pods per plant, pods per acre, seeds per plant, seeds per pod, weight per seed, weight per pod, pods per node, number of nodes, and the number of internodes per plant.

In still further embodiment, “sugarcane yield” can be measured as cane yield (tons per acre; kilograms per hectare), total recoverable sugar (pounds per ton), and sugar yield (tons per acre).

In yet still further embodiment, “wheat yield” can include: cereal per unit area, grain number, grain weight, grain size, grains per head, seeds per head, seeds per plant, heads per acre, number of viable tillers per plant, composition of seed (for example, carbohydrates, starch, oil, and protein) and characteristics of seed fill.

The terms “yield”, “seed yield” are defined above for a number of core crops. The terms “increased”, “improved”, “enhanced” are interchangeable and are defined herein.

In an embodiment, the present disclosure provides a method for the production of plants having increased yield. Performance of the method gives plants increased yield. “Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; (ii) increased early vigor, defined herein as an improved seedling aboveground area approximately three weeks post-germination. “Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor in corn, for example, is a combination of the ability of corn seeds to germinate and emerge after planting and the ability of the young corn plants to grow and develop after emergence. Plants having early vigor also show increased seedling survival and better establishment of the crop, which often results in highly uniform fields with the majority of the plants reaching the various stages of development at substantially the same time, which often results in increased yield. Therefore early vigor can be determined by measuring various factors, such as kernel weight, percentage germination, percentage emergence, seedling growth, seedling height, root length, root and shoot biomass, canopy size and color and others; (iii) increased total seed yield, which includes an increase in seed biomass (seed weight) and which can be an increase in the seed weight per plant or on an individual seed basis; increased number of panicles per plant; increased pods, increased number of nodes, increased number of flowers (“florets”) per panicle/plant; increased seed fill rate; increased number of filled seeds; increased seed size (length, width, area, perimeter), which can also influence the composition of seeds; increased seed volume, which can also influence the composition of seeds. Increased yield can also result in modified architecture, or can occur because of modified plant architecture; (iv) increased harvest index, which is expressed as a ratio of the yield of harvestable parts, such as seeds, over the total biomass; (v) increased kernel weight, which is extrapolated from the number of filled seeds counted and their total weight. An increased kernel weight can result from an increased seed size and/or seed weight, an increase in embryo size, endosperm size, aleurone and/or scutellum, or other parts of the seed; and (vi) increased ear biomass, which is the weight of the ear and can be represented on a per ear, per plant or per plot basis.

In one embodiment, increased yield can be increased seed yield, and is selected from one of the following: (i) increased seed weight; (ii) increased number of filled seeds; and (iii) increased harvest index.

The disclosure also extends to harvestable parts of a plant such as, but not limited to seeds, leaves, fruits, flowers, bolls, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example, seeds, or weight of seeds, per acre, pounds per acre, bushels per acre, tones per acre, tons per acre, kilo per hectare.

This disclosure further provides a method of increasing yield in a plant by crossing a plant comprising a recombinant DNA molecule of the present disclosure with itself, a second plant from the same plant line, a wild type plant, or a plant from a different line of plants to produce a seed. The seed of the resultant plant can be harvested from fertile plants and be used to grow progeny generations of plant(s) of this disclosure. In addition to direct transformation of a plant with a recombinant DNA, transgenic plants can be prepared by crossing a first plant having a recombinant DNA with a second plant lacking the DNA. For example, recombinant DNA can be introduced into a first plant line that is amenable to transformation to produce a transgenic plant which can be crossed with a second plant line to introgress the recombinant DNA into the second plant line. A transgenic plant with a recombinant DNA having the polynucleotide of this disclosure provides at least one enhanced trait of increased yield, increased nitrogen use efficiency or increased water use efficiency compared to a control plant. Genetic markers associated with recombinant DNA can be used to identify transgenic progeny that is homozygous for the desired recombinant DNA. Progeny plants carrying the recombinant DNA can be hack crossed into either parental or transgenic lines multiple times, for example usually 6 to 8 generations, to produce a progeny plant with substantially the same genotype as the one original transgenic parental line. The term “progeny” denotes the offspring of any generation of a parent plant prepared by the methods of this disclosure comprising the recombinant polynucleotides as described herein.

As used herein “nitrogen use efficiency” refers to the processes which lead to an increase in the plant's yield, biomass, vigor, and growth rate per nitrogen unit applied. The processes can include the uptake, assimilation, accumulation, signaling, sensing, re-translocation (within the plant) and use of nitrogen by the plant.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance, and can also improve crop quality and biochemical constituents of the seed such as protein yield and oil yield. The terms “increased nitrogen use efficiency”, “enhanced nitrogen use efficiency”, and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Plants are more susceptible to drought during flowering and seed development (the reproductive stages), as plant's resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant on the short term. The terms “increased water use efficiency”, “enhanced water use efficiency”, and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein “water deficit” refers to the conditions or environments that provide less than optimal amounts of water needed for adequate/successful growth and development of plants.

As used herein “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein a “polynucleotide” is a nucleic acid molecule comprising a plurality of polymerized nucleotides. A polynucleotide may be referred to as a nucleic acid, oligonucleotide, nucleotide, or any fragment thereof. In many instances, a polynucleotide encodes a polypeptide (or protein) or a domain or fragment thereof. Additionally, a polynucleotide can comprise a promoter, an intron, an enhancer region, a polyadenylation site, a translation initiation site, 5′ or 3′ untranslated regions, a reporter gene, a selectable marker, a scorable marker, or the like. A polynucleotide can be single-stranded or double-stranded DNA or RNA. A polynucleotide optionally comprises modified bases or a modified backbone. A polynucleotide can be, for example, genomic DNA or RNA, a transcript (such as an mRNA), a cDNA, a PCR product, a cloned DNA, a synthetic DNA or RNA, or the like. A polynucleotide can be combined with carbohydrate(s), lipid(s), protein(s), or other materials to perform a particular activity such as transformation or form a composition such as a peptide nucleic acid (PNA). A polynucleotide can comprise a sequence in either sense or antisense orientations. “Oligonucleotide” is substantially equivalent to the terms amplimer, primer, oligomer, element, target, and probe and is preferably single-stranded.

As used herein a “recombinant polynucleotide” or “recombinant DNA” is a polynucleotide that is not in its native state, for example, a polynucleotide comprises a series of nucleotides (represented as a nucleotide sequence) not found in nature, or a polynucleotide is in a context other than that in which it is naturally found; for example, separated from polynucleotides with which it typically is in proximity in nature, or adjacent (or contiguous with) polynucleotides with which it typically is not in proximity. The “recombinant polynucleotide” or “recombinant DNA” refers to polynucleotide or DNA which has been genetically engineered and constructed outside of a cell including DNA containing naturally occurring DNA or cDNA or synthetic DNA. For example, the polynucleotide at issue can be cloned into a vector, or otherwise recombined with one or more additional nucleic acids.

As used herein a “polypeptide” comprises a plurality of consecutive polymerized amino acid residues for example, at least about 15 consecutive polymerized amino acid residues. In many instances, a polypeptide comprises a series of polymerized amino acid residues that is a transcriptional regulator or a domain or portion or fragment thereof. Additionally, the polypeptide can comprise: (i) a localization domain; (ii) an activation domain; (iii) a repression domain; (iv) an oligomerization domain; (v) a protein-protein interaction domain; (vi) a DNA-binding domain; or the like. The polypeptide optionally comprises modified amino acid residues, naturally occurring amino acid residues not encoded by a codon, non-naturally occurring amino acid residues.

As used herein “protein” refers to a series of amino acids, oligopeptide, peptide, polypeptide or portions thereof whether naturally occurring or synthetic.

As used herein a “recombinant polypeptide” is a polypeptide produced by translation of a recombinant polynucleotide.

A “synthetic polypeptide” is a polypeptide created by consecutive polymerization of isolated amino acid residues using methods well known in the art.

Recombinant DNA constructs are assembled using methods well known to persons of ordinary skill in the art and typically comprise a promoter operably linked to DNA, the expression of which provides an enhanced agronomic trait. Other construct components can include additional regulatory elements, such as 5′ leaders and introns for enhancing transcription, 3′ untranslated regions (such as polyadenylation signals and sites), and DNA for transit or targeting or signal peptides. A “DNA construct” as used in the present disclosure comprises at least one expression cassette having a promoter operable in plant cells and a polynucleotide of the present disclosure encoding a protein or variant of a protein or fragment of a protein that is functionally defined to maintain activity in host cells including plant cells, plant parts, explants and plants. DNA constructs are made that contain various genetic elements necessary for the expression of noncoding and coding polynucleotides in plants. Promoters, leaders, enhancers, introns, transit or targeting or signal peptide sequences, 3′ transcriptional termination regions are genetic elements that can be operably linked in a DNA construct.

Percent identity describes the extent to which polynucleotides or protein segments are invariant in an alignment of sequences, for example nucleotide sequences or amino acid sequences. An alignment of sequences is created by manually aligning two sequences, for example a stated sequence, as provided herein, as a reference, and another sequence, to produce the highest number of matching elements, for example, individual nucleotides or amino acids, while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” (“% identity”) as used herein is the identity fraction times 100.

As used herein, a “functional fragment” refers to a portion of a polypeptide provided herein which retains full or partial molecular, physiological or biochemical function of the full length polypeptide. A functional fragment often contains the domain(s), such as Pfam domains, identified in the polypeptide provided in the sequence listing.

As used herein, a “homolog” or “homologues” means a protein in a group of proteins that perform the same biological function, for example, proteins that belong to the same Pfam protein family and that provide a common enhanced trait in transgenic plants of this disclosure. Homologs are expressed by homologous genes. With reference to homologous genes, homologs include orthologs, for example, genes expressed in different species that evolved from a common ancestral genes by speciation and encode proteins retain the same function, but do not include paralogs, for example, genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins have typically at least about 60% identity, in some instances at least about 70%, at least about 75%, at least about 80%, about 85%, at least about 90%, at least about 92%, at least about 94%, at least about 95%, at least about 96%, at least about 97%, at least about 98%, at least about 99%, and even at least about 99.5% identity over the full length of a protein identified as being associated with imparting an enhanced trait when expressed in plant cells. In one aspect of the disclosure homolog proteins have amino acid sequences that have at least about 80%, at least about 85%, at least about 90%, at least about 92%, at least about 94%, at least about 95%, at least about 96%, at least about 97%, at least about 98%, at least about 99%, and at least about 99.5% identity to a consensus amino acid sequence of proteins and homologs that can be built from sequences disclosed herein.

Homologs are inferred from sequence similarity, by comparison of protein sequences, for example, manually or by use of a computer-based tool using well-known sequence comparison algorithms such as BLAST and FASTA. A sequence search and local alignment program, for example, BLAST, can be used to search query protein sequences of a base organism against a database of protein sequences of various organisms, to find similar sequences, and the summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes. A further aspect of the homologs encoded by DNA useful in the transgenic plants of the invention are those proteins that differ from a disclosed protein as the result of deletion or insertion of one or more amino acids in a native sequence.

Other functional homolog proteins differ in one or more amino acids from those of a trait-improving protein disclosed herein as the result of one or more of the well-known conservative amino acid substitutions, for example, valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native protein or polypeptide can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side 30 chains is cysteine and methionine. Naturally conservative amino acids substitution groups are: valine-leucine, valine-isoleucine, phenylalanine-tyrosine, lysine-arginine, alaninevaline, aspartic acid-glutamic acid, and asparagine-glutamine. A further aspect of the disclosure includes proteins that differ in one or more amino acids from those of a described protein sequence as the result of deletion or insertion of one or more amino acids in a native sequence.

Homologs can be identified for the polypeptide sequences provided in Table 1, using the reciprocal search process as described in paragraph [0067]. The NCBI “blastp” program can be used for the sequence search, with E-value cutoff of 1e−4 to identify the initial significant hits. NCBI non-redundant amino-acid dataset can be used as the database of protein sequences of various organisms. Homologs with at least 95% identity over 95% of the length of the polypeptide sequences provided in Table 1 would be kept. From the sequences of the proteins identified in SEQ ID NOs: 2, 6 and 10, the corresponding homologous protein sequences as set forth as SEQ ID NOs: 13 (homolog of SEQ ID NO: 2), 14 (homolog of SEQ ID NO: 6), and SEQ ID NOs: 15, 16 and 17 (homologs of SEQ ID NO: 10), were identified for preparing additional transgenic seeds and plants with enhanced agronomic traits.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions or any desired combinations of such changes in a native polynucleotide or amino acid sequence.

With regard to polynucleotide variants, differences between presently disclosed polynucleotides and polynucleotide variants are limited so that the nucleotide sequences may be similar overall and, in many regions, identical. Due to the degeneracy of the genetic code, differences between the nucleotide sequences may be silent (for example, the amino acids encoded by the polynucleotide are the same, and the variant polynucleotide sequence encodes the same amino acid sequence as the presently disclosed polynucleotide). Variant nucleotide sequences can encode different amino acid sequences, in which case such nucleotide differences will result in amino acid substitutions, additions, deletions, insertions, truncations or fusions with respect to the similarly disclosed polynucleotide sequences. These variations can result in polynucleotide variants encoding polypeptides that share at least one functional characteristic. The degeneracy of the genetic code also dictates that many different variant polynucleotides can encode identical and/or substantially similar polypeptides.

As used herein “gene” or “gene sequence” refers to the partial or complete coding sequence of a gene, its complement, and its 5′ and/or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter can be subjected to subsequent processing such as chemical modification or folding to obtain a functional protein or polypeptide. By way of example, a transcriptional regulator gene encodes a transcriptional regulator polypeptide, which can be functional or require processing to function as an initiator of transcription.

As used herein, the term “promoter” refers generally to a DNA molecule that is involved in recognition and binding of RNA polymerase II and other proteins (trans-acting transcription factors) to initiate transcription. A promoter can be initially isolated from the 5′ untranslated region (5′ UTR) of a genomic copy of a gene. Alternately, promoters can be synthetically produced or manipulated DNA molecules. Promoters can also be chimeric, that is a promoter produced through the fusion of two or more heterologous DNA molecules. Plant promoters include promoter DNA obtained from plants, plant viruses, fungi and bacteria such as Agrobacterium and Bradyrhizobium bacteria.

Promoters which initiate transcription in all or most tissues of the plant are referred to as “constitutive” promoters. Promoters which initiate transcription during certain periods or stages of development are referred to as “developmental” promoters. Promoters whose expression is enhanced in certain tissues of the plant relative to other plant tissues are referred to as “tissue enhanced” or “tissue preferred” promoters. Promoters which express within a specific tissue of the plant, with little or no expression in other plant tissues are referred to as “tissue specific” promoters. A promoter that expresses in a certain cell type of the plant, for example a microspore mother cell, is referred to as a “cell type specific” promoter. An “inducible” promoter is a promoter in which transcription is initiated in response to an environmental stimulus such as cold, drought or light; or other stimuli such as wounding or chemical application. Many physiological and biochemical processes in plants exhibit endogenous rhythms with a period of about 24 hours. A “diurnal promoter” is a promoter which exhibits altered expression profiles under the control of a circadian oscillator. Diurnal regulation is subject to environmental inputs such as light and temperature and coordination by the circadian clock.

As used herein, the term “leader” refers to a DNA molecule isolated from the untranslated 5′ region (5′ UTR) of a genomic copy of a gene and is defined generally as a nucleotide segment between the transcription start site (TSS) and the protein coding sequence start site. Alternately, leaders can be synthetically produced or manipulated DNA elements. A leader can be used as a 5′ regulatory element for modulating expression of an operably linked transcribable polynucleotide molecule.

As used herein, the term “intron” refers to a DNA molecule that can be isolated or identified from the genomic copy of a gene and can be defined generally as a region spliced out during mRNA processing prior to translation. Alternately, an intron can be a synthetically produced or manipulated DNA element. An intron can contain enhancer elements that effect the transcription of operably linked genes. An intron can be used as a regulatory element for modulating expression of an operably linked transcribable polynucleotide molecule. A DNA construct can comprise an intron, and the intron may or may not be heterologous with respect to the transcribable polynucleotide molecule.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide. Unlike promoters, enhancer elements do not usually include a transcription start site (TSS) or TATA box or equivalent sequence. A promoter can naturally comprise one or more enhancer elements that affect the transcription of an operably linked polynucleotide. An isolated enhancer element can also be fused to a promoter to produce a chimeric promoter cis-element, which confers an aspect of the overall modulation of gene expression. A promoter or promoter fragment can comprise one or more enhancer elements that effect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element can function to bind transcription factors that regulate transcription. Some enhancer elements bind more than one transcription factor, and transcription factors can interact with different affinities with more than one enhancer domain.

Expression cassettes of this disclosure can include a “transit peptide” or “targeting peptide” or “signal peptide” molecule located either 5′ or 3′ to or within the gene(s). These terms generally refer to peptide molecules that when linked to a protein of interest directs the protein to a particular tissue, cell, subcellular location, or cell organelle. Examples include, but are not limited to, chloroplast transit peptides (CTPs), chloroplast targeting peptides, mitochondrial targeting peptides, nuclear targeting signals, nuclear exporting signals, vacuolar targeting peptides, vacuolar sorting peptides. For description of the use of chloroplast transit peptides see U.S. Pat. No. 5,188,642 and U.S. Pat. No. 5,728,925. For description of the transit peptide region of an Arabidopsis EPSPS gene see Klee, H. J. et al (MGG (1987) 210:437-442. Expression cassettes of this disclosure can also include an intron or introns. Expression cassettes of this disclosure can contain a DNA near the 3′ end of the cassette that acts as a signal to terminate transcription from a heterologous nucleic acid and that directs polyadenylation of the resultant mRNA. These are commonly referred to as “3′-untranslated regions” or “3′-non-coding sequences” or “3′-UTRs”. The “3′ non-translated sequences” means DNA sequences located downstream of a structural nucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation signal can be derived from a natural gene, from a variety of plant genes, or from ‘1’-DNA. An example of a polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989. Recombinant DNA constructs in this disclosure generally include a 3′ element that typically contains a polyadenylation signal and site. Well-known 3′ elements include those from Agrobacterium tumefaciens genes such as nos 3′, tml 3′, Um- 3′, tms 3′, ocs 3′, tr7 3′, for example disclosed in U.S. Pat. No. 6,090,627; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in US Patent Application Publication Nos. 2002/0192813 A1; and the pea (Pisum sativum) ribulose biphosphate carboxylase gene (rbs 3′), and 3′ elements from the genes within the host plant.

Expression cassettes of this disclosure can also contain one or more genes that encode selectable markers and confer resistance to a selective agent such as an antibiotic or a herbicide. A number of selectable marker genes are known in the art and can be used in the present disclosure: selectable marker genes conferring tolerance to antibiotics like kanamycin and paromomycin (nptII), hygromycin B (aph IV), spectinomycin (aadA), US Patent Publication No. US 2009/0138985 A1 and gentamycin (aac3 and aacC4) or tolerance to herbicides like glyphosate (for example, 5-enolpyruvylshikimate-3-phosphate synthase (EPSPS), U.S. Pat. No. 5,627,061; U.S. Pat. No. 5,633,435; U.S. Pat. No. 6,040,497; U.S. Pat. No. 5,094,945), sulfonyl herbicides (for example, acetohydroxyacid synthase or acetolactate synthase conferring tolerance to acetolactate synthase inhibitors such as sulfonylurea, imidazolinone, triazolopyrimidine, pyrimidyloxybenzoates and phthalide (U.S. Pat. No. 6,225,105; U.S. Pat. No. 5,767,366; U.S. Pat. No. 4,761,373; U.S. Pat. No. 5,633,437; U.S. Pat. No. 6,613,963; U.S. Pat. No. 5,013,659; U.S. Pat. No. 5,141,870; U.S. Pat. No. 5,378,824; U.S. Pat. No. 5,605,011)), bialaphos or phosphinothricin or derivatives (for example, phosphinothricin acetyltransferase (bar) tolerance to phosphinothricin or glufosinate (U.S. Pat. No. 5,646,024; U.S. Pat. No. 5,561,236; U.S. Pat. No. 5,276,268; U.S. Pat. No. 5,637,489; U.S. Pat. No. 5,273,894); dicamba (dicamba monooxygenase, US Patent Application Publications No. US 2003/0115626 A1), or sethoxydim (modified acetyl-coenzyme A carboxylase for conferring tolerance to cyclohexanedione (sethoxydim)), and aryloxyphenoxypropionate (haloxyfop, U.S. Pat. No. 6,414,222).

Transformation vectors of this disclosure can contain one or more “expression cassettes”, each comprising a native or non-native plant promoter operably linked to a polynucleotide sequence of interest, which is operably linked to a 3′ UTR termination signal, for expression in an appropriate host cell. It also typically comprises sequences required for proper translation of the polynucleotide or transgene. As used herein, the term “transgene” refers to a polynucleotide molecule artificially incorporated into a host cell's genome. Such a transgene can be heterologous to the host cell. The expression cassette comprising the nucleotide sequence of interest can be chimeric, meaning that at least one of its components is heterologous with respect to at least one of its other components. As used herein the term “chimeric” refers to a DNA molecule that is created from two or more genetically diverse sources, for example, a first molecule from one gene or organism and a second molecule from another gene or organism.

As used herein “operably linked” means the association of two or more DNA fragments in a recombinant DNA construct so that the function of one, for example, protein-encoding DNA, is controlled by the other, for example, a promoter.

As used herein “expressed” means produced, for example, a protein is expressed in a plant cell when its cognate DNA is transcribed to mRNA that is translated to the protein. An “expressed” protein can also include its truncated version (for example, N-terminal truncated, C-terminal truncated or internal truncated) as long as the truncated version maintains the same or similar functionality as the full length version.

Transgenic plants can comprise a stack of one or more polynucleotides disclosed herein resulting in the production of multiple polypeptide sequences. Transgenic plants comprising stacks of polynucleotides can be obtained by either or both of traditional breeding methods or through genetic engineering methods. These methods include, but are not limited to, crossing individual transgenic lines each comprising a polynucleotide of interest, transforming a transgenic plant comprising a first gene disclosed herein with a second gene, and co-transformation of genes into a single plant cell. Co-transformation of genes can be carried out using single transformation vectors comprising multiple genes or genes carried separately on multiple vectors.

Transgenic plants comprising or derived from plant cells of this disclosure transformed with recombinant DNA can be further enhanced with stacked traits, for example, a crop plant having an enhanced trait resulting from expression of DNA disclosed herein in combination with herbicide and/or pest resistance traits. For example, genes of the current disclosure can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects, or improved quality traits such as improved nutritional value. Herbicides for which transgenic plant tolerance has been demonstrated and the method of the present disclosure can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. No. 5,094,945; U.S. Pat. No. 5,627,061; U.S. Pat. No. 5,633,435 and U.S. Pat. No. 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in US Patent Application Publication No. US 2003/0083480 A1 also for imparting glyphosate tolerance; dicamba monooxygenase disclosed in US Patent Application Publication No. US 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtl) described in Misawa et al, (1993) Plant J. 4:833-840 and in Misawa et al, (1994) Plant J. 6:481-489 for norflurazon tolerance; a polynucleotide molecule encoding acetohydroxyacid synthase (AIIAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance as disclosed in U.S. Pat. No. 7,112,665; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and US Patent Application Publication No. US 2002/0112260. Molecules and methods for imparting insect/nematode/virus resistance are disclosed in U.S. Pat. No. 5,250,515; U.S. Pat. No. 5,880,275; U.S. Pat. No. 6,506,599; U.S. Pat. No. 5,986,175 and US Patent Application Publication No. US 2003/0150017 A1.

### Plant Cell Transformation Methods

Numerous methods for transforming chromosomes in a plant cell with recombinant DNA are known in the art and are used in methods of producing a transgenic plant cell and plant. Two effective methods for such transformation are Agrobacterium-mediated transformation and microprojectile bombardment-mediated transformation. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice). Agrobacterium-mediated transformation methods are described in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 5,846,797 (cotton); U.S. Pat. No. 6,384,301 (soybean), U.S. Pat. No. 7,026,528 (wheat) and U.S. Pat. No. 6,329,571 (rice), US Patent Application Publication No. US 2004/0087030 A1 (cotton), and US Patent Application Publication No. US 2001/0042257 A1 (sugar beet), all of which are incorporated herein by reference for enabling the production of transgenic plants. Transformation of plant material is practiced in tissue culture on nutrient media, for example, a mixture of nutrients that allow cells to grow in vitro. Recipient cell targets include, but are not limited to, meristem cells, shoot tips, hypocotyls, calli, immature or mature embryos, and gametic cells such as microspores, pollen, sperm and egg cells. Callus can be initiated from tissue sources including, but not limited to, immature or mature embryos, hypocotyls, seedling apical meristems, microspores and the like. Cells containing a transgenic nucleus are grown into transgenic plants.

In addition to direct transformation of a plant material with a recombinant DNA, a transgenic plant can be prepared by crossing a first plant comprising a recombinant DNA with a second plant lacking the recombinant DNA. For example, recombinant DNA can be introduced into a first plant line that is amenable to transformation, which can be crossed with a second plant line to introgress the recombinant DNA into the second plant line. A transgenic plant with recombinant DNA providing an enhanced trait, for example, enhanced yield, can be crossed with a transgenic plant line having another recombinant DNA that confers another trait, for example herbicide resistance or pest resistance, or enhanced water use efficiency, to produce progeny plants having recombinant DNA that confers both traits. Typically, in such breeding for combining traits the transgenic plant donating the additional trait is the male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, for example, marker identification by analysis for recombinant DNA or, in the case where a selectable marker is linked to the recombinant DNA, by application using a selective agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as the original transgenic parental line but for the recombinant DNA of the other transgenic parental line.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or a herbicide. Any of the herbicides to which plants of this disclosure can be resistant is a agent for selective markers. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroΛ or EPSPS). Examples of such selectable markers are illustrated in U.S. Pat. No. 5,550,318; U.S. Pat. No. 5,633,435; U.S. Pat. No. 5,780,708 and U.S. Pat. No. 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to a selective agent, or plant cells that have been scored positive in a screening assay, may be cultured in vitro to regenerate plantlets. Developing plantlets regenerated from transformed plant cells can be transferred to plant growth mix, and hardened off, for example, in an environmentally controlled chamber at about 85% relative humidity, 600 ppm CO₂, and 25-250 micro-einsteins m⁻²s⁻¹ of light, prior to transfer to a greenhouse or growth chamber for maturation. Plants are regenerated from about 6 weeks to 10 months after a transformant is identified, depending on the initial tissue, and plant species. Plants can be pollinated using conventional plant breeding methods known to those of skill in the art to produce seeds, for example self-pollination is commonly used with transgenic corn. The regenerated transformed plant or its progeny seed or plants can be tested for expression of the recombinant DNA and selected for the presence of an enhanced agronomic trait.

### Transgenic Plants and Seeds

Transgenic plants derived from transgenic plant cells having a transgenic nucleus of this disclosure are grown to generate transgenic plants having an enhanced trait as compared to a control plant, and produce transgenic seed and haploid pollen of this disclosure. Such plants with enhanced traits are identified by selection of transformed plants or progeny seed for the enhanced trait. For efficiency a selection method is designed to evaluate multiple transgenic plants (events) comprising the recombinant DNA, for example, multiple plants from 2 to 20 or more transgenic events. Transgenic plants grown from transgenic seeds provided herein demonstrate improved agronomic traits that contribute to increased yield or other traits that provide increased plant value, including, for example, improved seed quality. Of particular interest are plants having increased water use efficiency or drought tolerance, enhanced high temperature or cold tolerance, increased yield, increased nitrogen use efficiency.

Table 1 provides a list of protein-encoding DNA (“genes”) as recombinant DNA for production of transgenic plants with enhanced traits, the elements of Table 1 are described by reference to:

“NUC SEQ ID NO” which identifies a DNA sequence.

“PEP SEQ ID NO” which identifies an amino acid sequence.

“Gene ID” which refers to an arbitrary identifier.

“Protein Name” which is a common name for protein encoded by the recombinant DNA.

TABLE 1

| NUC | PEP | | |
|---|---|---|---|
| SEQ ID | SEQ ID | | |
| NO | NO | Gene ID | Protein Name |
| 1 | 2 | TRDX2-1 | Arabidopsis AP2 family transcription |
| | | | factor |
| 3 | 4 | TRDX2-2 | Soybean protein containing a SRF-type |
| | | | transcription factor domain |
| 5 | 6 | TRDX2-3 | Soybean auxin-induced protein aux22 |
| 7 | 8 | TRDX2-4 | Canola Myb domain protein DNA binding |
| | | | transcription factor |
| 9 | 10 | TRDX2-5 | Corn homolog of Rice Pi starvation- |
| | | | induced transcription factor 1 |
| 11 | 12 | TRDX2-6 | Arabidopsis homeobox-leucine zipper |
| | | | transcription factor 8 |

Selection Methods for Transgenic Plants with Enhanced Traits

Within a population of transgenic plants each regenerated from a plant cell with recombinant DNA many plants that survive to fertile transgenic plants that produce seeds and progeny plants will not exhibit an enhanced agronomic trait. Selection from the population is necessary to identify one or more transgenic plants with an enhanced trait. Transgenic plants having enhanced traits are selected from populations of plants regenerated or derived from plant cells transformed as described herein by evaluating the plants in a variety of assays to detect an enhanced trait, for example, increased water use efficiency or drought tolerance, enhanced high temperature or cold tolerance, increased yield, increased nitrogen use efficiency, enhanced seed composition such as enhanced seed protein and enhanced seed oil. These assays can take many forms including, but not limited to, direct screening for the trait in a greenhouse or field trial or by screening for a surrogate trait. Such analyses can be directed to detecting changes in the chemical composition, biomass, physiological property, or morphology of the plant. Changes in chemical compositions such as nutritional composition of grain can be detected by analysis of the seed composition and content of protein, free amino acids, oil, free fatty acids, starch or tocopherols. Changes in chemical compositions can also be detected by analysis of contents in leaves, such as chlorophyll or carotenoid contents. Changes in biomass characteristics can be evaluated on greenhouse or field grown plants and can include plant height, stem diameter, root and shoot dry weights, canopy size; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant to appear to be a normal plant as compared to changes toward bushy, taller, thicker, narrower leaves, striped leaves, knotted trait, chlorosis, albino, anthocyanin production, or altered tassels, cars or roots. Other selection properties include days to pollen shed, days to silking, leaf extension rate, chlorophyll content, leaf temperature, stand, seedling vigor, internode length, plant height, leaf number, leaf area, tillering, brace roots, stay green or delayed senescence, stalk lodging, root lodging, plant health, barreness/prolificacy, green snap, and pest resistance. In addition, phenotypic characteristics of harvested grain can be evaluated, including number of kernels per row on the ear, number of rows of kernels on the ear, kernel abortion, kernel weight, kernel size, kernel density, ear biomass and physical grain quality.

Assays for screening for a desired trait are readily designed by those practicing in the art. The following illustrates screening assays for corn traits using hybrid corn plants. The assays can be readily adapted for screening other plants such as canola, wheat, cotton and soybean either as hybrids or inbreds.

Transgenic corn plants having increased nitrogen use efficiency can be identified by screening transgenic plants in the field under the same and sufficient amount of nitrogen supply as compared to control plants, where such plants provide higher yield as compared to control plants. Transgenic corn plants having increased nitrogen use efficiency can be identified where such plants provide higher yield as compared to control plants under the same nitrogen limiting conditions. For example, transgenic corn plants are show to have increased nitrogen use efficiency compared to control plants in Table 2.

Transgenic corn plants having increased yield are identified by screening progenies of the transgenic plants over multiple locations for several years with plants grown under optimal production management practices and maximum weed and pest control. Selection methods can be applied in multiple and diverse geographic locations, for example up to 16 or more locations, over one or more planting seasons, for example at least two planting seasons, to statistically distinguish yield improvement from natural environmental effects.

Transgenic corn plants having increased water use efficiency or drought tolerance are identified by screening plants in an assay where water is withheld for a period to induce stress followed by watering to revive the plants. For example, a selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

Although the plant cells and methods of this disclosure can be applied to any plant cell, plant, seed or pollen, for example, any fruit, vegetable, grass, tree or ornamental plant, the various aspects of the disclosure are applied to corn, soybean, cotton, canola, rice, barley, oat, wheat, turf grass, alfalfa, sugar beet, sunflower, quinoa and sugar cane plants.

The following examples are included to demonstrate aspects of the disclosure. Those of skill in the art should, in light of the present disclosure, appreciate that many changes can be made in the specific aspects which are disclosed and still obtain a like or similar results without departing from the spirit and scope of the disclosure.

### Example 1

### Corn Transformation

This example illustrates transformation methods in producing a transgenic corn plant cell, plant, and seed having an enhanced trait, for example, increased water use efficiency or drought tolerance, increased yield, and increased nitrogen use efficiency as shown in Tables 2, 3, 4 and 7.

For Agrobacterium-mediated transformation of corn embryo cells corn plants were grown in the greenhouse and cars were harvested when the embryos were 1.5 to 2.0 mm in length. Ears were surface-sterilized by spraying or soaking the ears in 80% ethanol, followed by air drying. Immature embryos were isolated from individual kernels on surface-sterilized ears. Shortly after excision, immature maize embryos were inoculated with overnight grown Agrobacterium cells, and incubated at room temperature with Agrobacterium for 5-20 minutes. Inoculated immature embryos were then co-cultured with Agrobacterium for 1 to 3 days at 23° C. in the dark. Co-cultured embryos were transferred to selection media and cultured for approximately two weeks to allow embryogenic callus to develop. Embryogenic calli were transferred to culture medium containing glyphosate and subcultured at about two week intervals. Transformed plant cells were recovered 6 to 8 weeks after initiation of selection.

For Agrobacterium-mediated transformation of maize callus immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media. Paromomycin resistant calli are identified about 6-8 weeks after initiation of selection.

To regenerate transgenic corn plants individual transgenic calli resulting from transformation and selection were placed on media to initiate shoot and root development into plantlets. Plantlets were transferred to potting soil for initial growth in a growth chamber at 26° C. followed by a mist bench before transplanting to 5 inch pots where plants were grown to maturity. The regenerated plants were self-fertilized and seeds were harvested for use in one or more methods to select seeds, seedlings or progeny second generation transgenic plants (R2 plants) or hybrids, for example, by selecting transgenic plants exhibiting an enhanced trait as compared to a control plant.

The above process can be repeated to produce multiple events of transgenic corn plants from cells that were transformed with recombinant DNA from the genes identified in Table 1. Progeny transgenic plants and seeds of the transformed plants were screened for the presence and single copy of the inserted gene, and for increased yield, increased nitrogen use efficiency, and increased water use efficiency as shown in Tables 2, 3, 4 and 7. From each group of multiple events of transgenic plants with a specific recombinant DNA from Table 1 the event(s) that showed increased yield, increased nitrogen use efficiency, and increased water use efficiency was (were) identified.

### Example 2

### Soybean Transformation

This example illustrates plant transformation in producing a transgenic soybean plant cell, plant, and seed having an enhanced trait, for example, increased yield, increased nitrogen use efficiency and increased water use efficiency. An example for increased yield in soybean is shown in Table 5.

For Agrobacterium mediated transformation, soybean seeds were imbibed overnight and the meristem explants excised. Soybean explants were mixed with induced Agrobacterium cells containing plasmid DNA with the gene of interest cassette and a plant selectable marker cassette no later than 14 hours from the time of initiation of seed imbibition, and wounded using sonication. Following wounding, explants were placed in co-culture for 2-5 days at which point they were transferred to selection media to allow selection and growth of transgenic shoots. Resistant shoots were harvested in approximately 6-8 weeks and placed into selective rooting media for 2-3 weeks. Shoots producing roots were transferred to the greenhouse and potted in soil. Shoots that remained healthy on selection, but did not produce roots were transferred to non-selective rooting media for an additional two weeks. Roots from any shoots that produced roots off selection were tested for expression of the plant selectable marker before they were transferred to the greenhouse and potted in soil.

The above process can be repeated to produce multiple events of transgenic soybean plants from cells that were transformed with recombinant DNA from the genes identified in Table 1. Progeny transgenic plants and seed of the transformed plant cells were screened for the presence and single copy of the inserted gene, and for increased increased yield, increased nitrogen use efficiency and increased water use efficiency.

### Example 3

### Canola Transformation

This example illustrates plant transformation in producing the transgenic canola plants of this disclosure and the production and identification of transgenic seed for transgenic canola having increased yield, increased nitrogen use efficiency and increased water use efficiency.

Tissues from in vitro grown canola seedlings were prepared and inoculated with overnight-grown Agrobacterium cells containing plasmid DNA with a gene of interest cassette and a plant selectable marker cassette. Following co-cultivation with Agrobacterium, the infected tissues were allowed to grow on selection to promote growth of transgenic shoots, followed by growth of roots from the transgenic shoots. The selected plantlets were then transferred to the greenhouse and potted in soil. Molecular characterizations were performed to confirm the presence of the gene of interest, and its expression in transgenic plants and progenies. Progeny transgenic plants were selected from a population of transgenic canola events under specified growing conditions and were compared with control canola plants.

The above process can be repealed to produce multiple events of transgenic canola plants from cells that were transformed with recombinant DNA from the genes identified in Table 1. Progeny transgenic plants and seed of the transformed plant cells were screened for the presence and single copy of the inserted gene, and for increased water use efficiency, increased yield, and increased nitrogen use efficiency. From each group of multiple events of transgenic plants with a specific recombinant DNA from Table 1 the event(s) that showed increased yield, increased water use efficiency and increased nitrogen use efficiency was (were) identified.

### Example 4

### Phenotypic Evaluation of Transgenic Plants for Increased Nitrogen Use Efficiency

Corn nitrogen field efficacy trials were conducted to identify genes that can improve nitrogen use efficiency under nitrogen limiting conditions leading to increased yield performance as compared to non transgenic controls. A yield increase in corn can be manifested as one or more of the following: an increase in the number of ears per plant, an increase in the number of rows, number of kernels per row, kernel weight, thousand kernel weight, ear length/diameter/weight or ear biomass, ear biomass per plot, increase in the seed filling rate (which is the number of filled seeds divided by the total number of seeds and multiplied by 100), among others. In a first trial, each field was planted under nitrogen limiting condition (60 lbs/acre) and the dry weight of corn ears was compared to control plants to measure the yield increases. In a second trial, each field was planted under nitrogen limiting condition (60 lbs/acre) and the fresh weight of corn ears was compared to control plants to measure the yield increases.

Table 2 provides an example of a protein encoding DNA or polynucleotide sequence (“gene”) for producing transgenic corn plant with increased nitrogen use efficiency as compared to a control plant. Polynucleotide sequences in constructs with at least one event showing significant yield increase across multiple locations at p≦0.2 are included. The elements of Table 2 are described by reference to:

“SEQ ID NO: Polynucleotide” which identifies a nucleotide sequence from SEQ ID NO: 3.

“SEQ ID NO: Polypeptide” which identifies an amino acid sequence from SEQ ID NO: 4.

“Gene identifier” which refers to an arbitrary identifier.

“NUE results” which represents the result of nitrogen field trial for plants comprising a sequence in a construct with at least one event showing significant yield increase at p≦0.2 across locations. The first number refers to the number of events with significant yield increase, whereas the second number refers to the total number of events tested for each sequence in the construct. The NUE results for gene TRDX2-2 were derived from seven total events from one trial.

TABLE 2
Recombinant DNA for increased nitrogen use efficiency in corn

| SEQ ID NO: | SEQ ID NO: | Gene | NUE |
|---|---|---|---|
| Polynucleotide | Polypeptide | Identifier | Results |
| 3 | 4 | TRDX2-2 | 4/7 |

### Example 5

### Phenotypic Evaluation of Transgenic Plants for Increased Yield

This example illustrates selection and identification of transgenic plants for increased yield in both dicotyledonous and monocotyledonous plants with primary examples presented for corn, soybean, and canola in Table 3, 5 and 6 respectively. Polynucleotide sequences in constructs with at least one event that resulted in significant yield increase across locations at p≦0.2 are included.

Selection of Transgenic Plants for Increased Yield.

Effective selection of increased and/or enhanced yielding transgenic plants uses hybrid progenies of the transgenic plants for corn, cotton, and canola, or inbred progenies of transgenic plants for soybean plants plant such as corn, cotton, canola, or inbred plant such as soy, canola and cotton over multiple locations with plants grown under optimal production management practices. An exemplary target for improved yield is a 2% to 10% increase in yield as compared to yield produced by plants grown from seed of a control plant. Selection methods can be applied in multiple and diverse geographic locations, for example up to 16 or more locations, over one or more planting seasons, for example at least two planting seasons, to statistically distinguish yield improvement from natural environmental effects.

Increased Yield in Corn

Table 3 provides a list of protein encoding DNA or polynucleotide sequences (“genes”) in the production of transgenic corn plants with increased yield as compared to a control plant. The elements of Table 3 are described by reference to:

“SEQ ID NO: Polynucleotide” which identifies a nucleotide sequence.

“SEQ ID NO: Polypeptide” which identifies an amino acid sequence.

“Gene identifier” which refers to an arbitrary identifier.

“Broad acre yield results” represent results from broad acre yield field trials for plants comprising the sequence in a construct with at least one event showing significant yield increase at p≦0.2 across locations. The first number refers to the number of events with significant yield increase, whereas the second number refers to the total number of events tested for each sequence in a construct. For example, as indicated in Table 3, gene for TRDX2-1, TRDX2-5 and TRDX2-6 resulted in significantly positive yield results in at least one event across locations at a p≦0.2 in one broad acre yield trial and gene TRDX2-2 was tested in two broad acre yield trials resulted in 1 of 7 total events yield positive in trial 1 and 1 of 10 total events yield positive in trial 2 compared to non-transgenic control plants.

TABLE 3
Recombinant DNA for increased yield in corn

| | | | | Broad Acre |
|---|---|---|---|---|
| | | | Broad Acre | Yield |
| SEQ ID NO: | SEQ ID NO: | Gene | Yield Results | Results |
| Polynucleotide | Polypeptide | Identifier | Trial 1 | Trial 2 |
| 1 | 2 | TRDX2-1 | 3/8 | — |
| 3 | 4 | TRDX2-2 | 1/7 | 1/10 |
| 9 | 10 | TRDX2-5 | 1/4 | — |
| 11 | 12 | TRDX2-6 | 1/8 | — |

A yield increase in corn can also be manifested as an increase in corn ear biomass which can be calculated on a per plant or per plot basis using a determination for weight per ear for any number of events in a plot compared to a control. This example illustrates the selection of a corn plant with increased yield measured as an increase in ear biomass.

Corn car biomass was measured for plants grown under high planting density (52,000 plants per acre), nitrogen limiting conditions of 60 pounds (lbs) per acre or water limiting conditions (chronic drought condition).

A correction factor was applied to achieve a corrected plot ear biomass that was used to correct for ear biomass if there was a discrepancy in the number of plants per plot. To apply a corrected value to plot ear biomass, an estimate of plot ear biomass was measured in the full field trials, which was determined on a field by field location basis for plot ear biomass and stand. This analysis for ear biomass derived from the full field trials was used to calculate a correction factor that effectively reduced and accounted for the projected ear biomass per plot (fresh ear weight per plot basis). The factor for corrected ear biomass was applied to the plots and used to provide ear biomass per plot in the density, NUE and WUE trials.

The change or delta between transgenic events and non-transgenic control events for ear biomass in a plot was used to calculate a percent change for plot ear biomass.

“Corn ear biomass” was used as a parameter to predict increased yield for an individual event on a per plot basis. Table 4 presents events positive for corn ear biomass for plants comprising the sequences in constructs with at least one event showing significant increase in ear biomass or fresh weight per plot at a significant p≦0.2 across three locations. The ears were individually collected and ear biomass was measured by taking a fresh weight on the corn ear, which was the mass (grams) of the non-shelled whole ear (grain+cob) at measured at a physiological maturity stage of R6. Corn ear biomass per plot was used as an estimate of predicted yield increase in the field and was determined for each transgenic event in a construct as compared to non-transgenic wild-type control plants. The positive events for ear biomass are reported with the number of events with significant increase in ear biomass (first number N/N) compared to the total number of plants tested for each event (second number N/N). The screens for density and NUE resulted in positive events which met the statistical criteria for significance across locations at p≦0.2 across three locations and are reported in Table 4.

Table 4 provides a reference to:

“SEQ ID NO: Polynucleotide” which identifies a nucleotide sequence.

“SEQ ID NO: Polypeptide” which identifies an amino acid sequence.

“Gene identifier” which refers to an arbitrary identifier.

“Event” which refers to an individual event for a given construct.

“Density” refers to a spacing of plants to estimate a field density of 52,000 plants per acre.

“NUE” refers to nitrogen use efficiency or increased yield under nitrogen limiting conditions of 60 pounds (lbs) nitrogen applied per acre.

TABLE 4
Recombinant DNA for increased corn ear biomass [NS = non-statistically
significant]

| SEQ ID NO: | SEQ ID NO: | Gene | | | |
|---|---|---|---|---|---|
| Polynucleotide | Polypeptide | Identifier | Event | Density | NUE |
| 9 | 10 | TRDX2-5 | 1 | NS | 2/4 |
| 11 | 12 | TRDX2-6 | 1 | 3/4 | NS |
| 11 | 12 | TRDX2-6 | 2 | 3/4 | NS |

Increased Yield in Soybean

A yield increase in soybean can be manifested as one or more of the following: an increase in pods per plant, pods per acre, seeds per plant, seeds per pod, weight per seed, weight per pod, pods per node, number of nodes, and the number of internodes per plant.

Table 5 provides a list of protein encoding DNA or polynucleotide sequences used (“genes”) in the production of transgenic soybean plants with increased yield as compared to a control plant. The elements of Table 5 are described by reference to:

“SEQ ID NO: Polynucleotide” which identifies a nucleotide sequence.

“SEQ ID NO: Polypeptide” which identifies an amino acid sequence.

“Gene identifier” which refers to an arbitrary identifier.

“Broad acre yield results” which refers to the sequence in a construct with at least one event showing significant yield increase at p≦0.2 across locations. The first number refers to the number of events with significant yield increase, whereas the second number refers to the total number of events tested for each sequence in a construct. As indicated in Table 5, gene TRDX2-3 was tested in two broad acre yield trials with 4 of 10 total events in trial 1 and 1 of 4 total events in trial 2 resulted in significantly positive yield compared to non-transgenic control plants.

TABLE 5
Recombinant DNA for increased yield in soybean

| | | | Broad | Broad |
|---|---|---|---|---|
| | | | Acre Yield | Acre Yield |
| SEQ ID NO: | SEQ ID NO: | Gene | Results | Results |
| Polynucleotide | Polypeptide | Identifier | Trial 1 | Trial 2 |
| 5 | 6 | TRDX2-3 | 4/10 | 1/4 |

Increased Yield in Canola

A yield increase in canola can be manifested as one or more of the following: an increase in silique number, number of sliques per plant, number of siliques per node, number of internodes, incidence of silique shatter, seeds per silique, seed weight per silique, improved seed, oil, or protein composition.

Table 6 provides a list of protein encoding DNA or polynucleotide sequences used (“genes”) in the production of transgenic canola plants with increased yield as compared to a control plant. The elements of Table 6 are described by reference to:

“SEQ ID NO: Polynucleotide” which identifies a nucleotide sequence.

“SEQ ID NO: Polypeptide” which identifies an amino acid sequence.

Gene identifier” which refers to an arbitrary identifier.

“Broad acre yield results” which refers to the sequence in a construct with at least one event showing significant yield increase at p≦0.2 across locations. The first number refers to the number of events with significant yield increase, whereas the second number refers to the total number of events tested for each sequence in a construct.

TABLE 6
Recombinant DNA for increased Yield in Canola

| | | | Broad |
|---|---|---|---|
| | | | Acre |
| SEQ ID NO: | SEQ ID NO: | Gene | Yield |
| Polynucleotide | Polypeptide | Identifier | Results |
| 7 | 8 | TRDX2-4 | 1/8 |

### Example 6

### Phenotypic Evaluation of Transgenic Plants for Increased Water Use Efficiency

Transgenic corn plants having increased water use efficiency or drought tolerance. Corn field trials were conducted to identify genes for increased yield under standard field conditions, and for improved water use efficiency under drought stress conditions leading to increased yield performance as compared to non transgenic controls. Before planting, all fields had sufficient water for germination but limited enough to allow soil to dry adequately at the time of stress imposition. Evapotranspiration (ET) was calculated from data provided by a site-specific weather station or the nearest/most accurate available weather station when compared to the trial site. Field trials under standard conditions were managed with ET replacement, which was implanted based on the trials under drought stress conditions, and soil moisture during stressed period. Irrigation frequency depended on the specific site, irrigation method and soil type. Trial locations under drought conditions were characterized based on stress severity. Corn yield was measured to determine yield increases as compared to control plants.

Table 7 provides an example of protein encoding DNA or polynucleotide sequences (“genes”) for producing transgenic corn plant with increased water use efficiency as compared to a control plant. Polynucleotide sequences in constructs with at least one event showing significant yield increase across multiple locations at p≦0.2 are included. The elements of Table 7 are described by reference to:

“SEQ ID NO: Polynucleotide” which identifies a nucleotide sequence from SEQ 11) NO: 3.

“SEQ ID NO: Polypeptide” which identifies an amino acid sequence from SEQ ID NO: 4.

“Gene identifier” which refers to an arbitrary identifier.

“WUE results” which refers to the sequence in a construct with at least one event showing significant yield increase at p≦0.2 across locations. The first number refers to the number of events with significant yield increase under water limiting conditions, whereas the second number refers to the total number of events tested for each sequence in the construct. The WUE results for gene TRDX2-2 were derived from seven total events with four of which resulted in significantly positive yield increases from the trial.

TABLE 7
Recombinant DNA for increased water use efficiency in corn

| SEQ ID NO: | SEQ ID NO: | Gene | WUE |
|---|---|---|---|
| polynucleotide | polypeptide | Identifier | Results |
| 3 | 4 | TRDX2-2 | 4/7 |

We claimed:

1. A plant comprising a recombinant DNA molecule comprising a polynucleotide encoding a polypeptide, wherein the nucleotide sequence of the polynucleotide is selected from the group consisting of:
a) a nucleotide sequence encoding a protein having the amino acid sequence of SEQ ID NO: 2, 4, 6, 8, 10, 12, 13, 14, 15, 16 or 17; and
b) a nucleotide sequence encoding a protein with at least 90%, at least 91%, at least 92%, at least 93%, at least 94%, at least 95%, at least 96%, at least 97%, at least 98%, at least 99% identity to SEQ ID NO: 2, 4, 6, 8, 10, 12, 13, 14, 15, 16 or 17; and
wherein said plant has at least one enhanced trait as compared to a control plant.

2. The plant of claim 1, wherein said enhanced trait is selected from the group consisting of increased yield, increased nitrogen use efficiency and increased water use efficiency.

3. The plant of claim 1, wherein said plant is a monocot plant or is a member of the family Poaceae, wheat plant, maize plant, sweet corn plant, rice plant, wild rice plant, barley plant, rye, millet plant, sorghum plant, sugar cane plant, turfgrass plant, bamboo plant, oat plant, brome-grass plant, Miscanthus plant, pampas grass plant, switchgrass (Panicum) plant, and/or teosinte plant, or is a member of the family Alliaceae, onion plant, leek plant, garlic plant; or wherein the plant is a dicot plant or is a member of the family Amaranthaceae, spinach plant, quinoa plant, a member of the family Anacardiaceae, mango plant, a member of the family Asteraceae, sunflower plant, endive plant, lettuce plant, artichoke plant, a member of the family Brassicaceae, Arabidopsis thaliana plant, rape plant, oilseed rape plant, broccoli plant, Brussels sprouts plant, cabbage plant, canola plant, cauliflower plant, kohlrabi plant, turnip plant, radish plant, a member of the family Bromeliaceae, pineapple plant, a member of the family Caricaceae, papaya plant, a member of the family Chenopodiaceae, beet plant, a member of the family Curcurbitaceae, melon plant, cantaloupe plant, squash plant, watermelon plant, honeydew plant, cucumber plant, pumpkin plant, a member of the family Dioscoreaceae, yam plant, a member of the family Ericaceae, blueberry plant, a member of the family Euphorbiaceae, cassava plant, a member of the family Fabaceae, alfalfa plant, clover plant, peanut plant, a member of the family Grossulariaceae, currant plant, a member of the family Juglandaceae, walnut plant, a member of the family Lamiaceae, mint plant, a member of the family Lauraceae, avocado plant, a member of the family Leguminosae, soybean plant, bean plant, pea plant, a member of the family Malvaceae, cotton plant, a member of the family Marantaceae, arrowroot plant, a member of the family Myrtaceae, guava plant, eucalyptus plant, a member of the family Rosaceae, peach plant, apple plant, cherry plant, plum plant, pear plant, prune plant, blackberry plant, raspberry plant, strawberry plant, a member of the family Rubiaceae, coffee plant, a member of the family Rutaccae, citrus plant, orange plant, lemon plant, grapefruit plant, tangerine plant, a member of the family Salicaceae, poplar plant, willow plant, a member of the family Solanaceae, potato plant, sweet potato plant, tomato plant, Capsicum plant, tobacco plant, tomatillo plant, eggplant plant, Atropa belladona plant, Datura stramonium plant, a member of the family Vitaceae, grape plant, a member of the family Umbelliferae, carrot plant, or a member of the family Musaceae; or wherein the plant is a member of the family Pinaceae, cedar plant, fir plant, hemlock plant, larch plant, pine plant, or spruce plant.

4. The plant of claim 1, wherein the recombinant DNA molecule further comprises a promoter that is operably linked to the polynucleotide encoding a polypeptide, wherein said promoter is selected from the group consisting of a constitutive, inducible, tissue specific, diurnally regulated, tissue enhanced, and cell specific promoter.

5. The plant of claim 1, wherein said plant is selected from the group consisting of corn, soybean, cotton, canola, rice, barley, oat, wheat, turf grass, alfalfa, sugar beet, sunflower, quinoa and sugar cane.

6. The plant of claim 1, wherein said plant is a propagule selected from the group consisting of a cell, pollen, ovule, flower, embryo, leaf, root, stem, shoot, meristem, grain and seed.

7. A method for producing a plant comprising:
introducing into a plant cell a recombinant DNA molecule comprising a polynucleotide encoding a polypeptide, wherein the nucleotide sequence of the polynucleotide is selected from the group consisting of:
a) a nucleotide sequence encoding a protein having the amino acid sequence of SEQ ID NO: 2, 4, 6, 8, 10, 12, 13, 14, 15, 16, or 17; and
b) a nucleotide sequence encoding a protein with at least 90%, at least 91%, at least 92%, at least 93%, at least 94%, at least 95%, at least 96%, at least 97%, at least 98%, at least 99% identity to SEQ ID NO: 2, 4, 6, 8, 10, 12, 13, 14, 15, 16, or 17; and
growing a plant from said plant cell.

8. The method of claim 7, further comprising selecting the plant comprising said recombinant DNA molecule with at least one enhanced trait selected from the group consisting of increased yield, increased nitrogen use efficiency and increased water use efficiency as compared to a control plant.

9. A method for increasing yield, increasing nitrogen use efficiency, or increasing water use efficiency in a plant comprising:
a) crossing the plant of claim 1 with itself, a second plant from the same plant line, a wild type plant, or a second plant from a different line of plants to produce a seed;
b) growing said seed to produce a plurality of progeny plants; and
c) selecting a progeny plant with increased yield, increased nitrogen use efficiency, or increased water use efficiency compared to a plant not having said recombinant DNA molecule.
